(19) United States
(12) Patent Application Publication
(10) Pub. No.: US 2026/0192098 A1
(43) Pub. Date: Jul. 9, 2026

(54) SONOHYSTEROGRAM CATHETER DEVICE

(71) Applicant: Fefelova Medicine Professional Corporation, Barrie (CA)

(72) Inventor: Evgeniya FEFELOVA, Barrie (CA)

(21) Appl. No.: 19/442,904

(22) Filed: Jan. 7, 2026

Related U.S. Application Data
(60) Provisional application No. 63/742,653, filed on Jan. 7, 2025.

(51) Int. Cl. A61M 31/00; A61M 25/00

(52) U.S. Cl. CPC A61M 31/005; A61M 25/0069; A61M 2210/1425; A61M 2210/1433

(57) ABSTRACT

Methods and devices are provided for delivering a fluid into a uterine cavity of a female reproductive system. More particularly, the present disclosure describes a sonohysterogram catheter device comprising a catheter tube and a shape-retentive annular sealing member surrounding a portion of the catheter tube. In examples, the catheter tube and sealing member are cooperatively configured such that when a first end of the catheter tube is positioned within a cervical canal of a female reproductive system, an exterior surface of the sealing member contacts tissue surrounding the cervical canal to occlude the cervical canal, with effect that fluid within the uterine cavity does not escape.

### RELATED APPLICATIONS

This application claims the benefit and priority of United States Provisional Patent Application No. 63/742653, filed January 7, 2025, the contents of which are incorporated herein by reference.

### FIELD

The present disclosure is related to methods and devices for intra-uterine catheters, and particularly, sonohysterogram catheter devices for the delivery of fluid for sonography procedures.

### BACKGROUND

A sonohysterogram is a gynecological diagnostic procedure that provides ultrasound imaging of the uterine cavity and patency of the Fallopian tubes. To improve the ultrasound imaging details, a fluid such as normal saline or contrast media (e.g., ExEm® Foam, Echovist®) may be injected into the uterus during the procedure. During fluid injection, it is desirable for the cervical opening to be occluded, to prevent leakage of the fluid from the uterus through the cervical opening.

Currently, a sonogram catheter is a commonly used device for delivering the fluid to the uterus and Fallopian tubes during sonohysterogram procedures. A conventional sonogram catheter includes a thin flexible tube coupled to a syringe at one end and having a small balloon that is inflated inside the uterine cavity, after placement through the external cervical OS opening and into the cervical canal, to occlude the cervical opening. However, existing sonogram catheters are expensive (particularly for clinics performing many sonohysterogram procedures daily) and have been reported to cause discomfort to the patient when the sonogram catheter is inserted into the uterine cavity. Furthermore, once inflated in the uterine cavity, the sonogram balloon can obscure the view of the uterine cavity, therefore hindering the capture of clear ultrasound images.

Accordingly, improvements to intra-uterine catheter devices are desired.

### SUMMARY

Methods and devices are provided for delivering a fluid into a uterine cavity of a female reproductive system. More particularly, in one example aspect, the present disclosure describes a sonohysterogram catheter device comprising a catheter tube and a shape-retentive annular sealing member surrounding a portion of the catheter tube. In examples, the catheter tube and sealing member are cooperatively configured such that when a first end of the catheter tube is positioned within a cervical canal of a female reproductive system, an exterior surface of the sealing member contacts tissue surrounding the cervical canal to occlude the cervical canal, with effect that fluid within the uterine cavity does not escape.

According to a first example aspect, a sonohysterogram catheter device is disclosed that includes a catheter tube defining a lumen that extends a length of the catheter tube from a fluid receiving port at a fluid receiving end of the catheter tube to a fluid delivery port at a fluid delivery end of the catheter tube. An annular sealing member is located proximate the fluid delivery end of the catheter tube such that when the fluid delivery end of the catheter tube is positioned within a cervical canal of a female reproductive system, an exterior surface of the annular sealing member contacts tissue surrounding the cervical canal to occlude the cervical canal and prevent fluid from escaping a uterine cavity of the female reproductive system. In some examples, the catheter tube comprises an elongated tubular body portion extending from the fluid receiving end to a back end of the annular sealing member and a tip extending from a front end of the annular sealing member to form the fluid delivery end, the elongated tubular body portion, annular sealing member and tip collectively defining the lumen.

In some examples, the annular sealing member and the tip are collectively configured such that the fluid delivery end will be positioned within the cervical canal when the exterior surface of the annular sealing member occludes the cervical canal.

In some examples, the elongated tubular body portion, annular sealing member and tip are molded as a unitary rigid or semi-rigid structure from a polymer material.

In some examples, the annular sealing member tapers generally from a larger diameter at the back end of the annular sealing member to a smaller diameter at the front end of the annular sealing member and includes a plurality of annular ridges that decrease in an outer diameter size from the back end to the front end.

In some examples, the outer diameter of the tip tapers from a larger outer diameter at a back end of the tip to a smaller outer diameter at the fluid delivery port.

In some examples, a length of the tip from the annular sealing member to the fluid delivery port is equal to or less than 20mm.

In some examples, the elongated tubular body portion is deformable to form a curve along a length thereof.

In some examples, one or more thread elements are formed on an outer circumference of the elongated tubular body proximate the fluid receiving end to enable a fluid container to be secured thereto to deliver a fluid from the fluid container to the lumen via the fluid receiving port.

According to a further example aspect, a method is disclosed for using the sonohysterogram catheter device for delivering fluid into a uterine cavity of a female reproductive system. The method includes: positioning the catheter tube of the sonohysterogram catheter device along a vaginal tract of the female reproductive system and in abutment with the tissue surrounding the cervical canal of the female reproductive system; applying an abutment pressure to the sonohysterogram catheter device for providing a fluid seal between the sealing member and the tissue surrounding the cervical canal; and expelling the fluid from a container secured to the fluid receiving port at the fluid receiving end of the catheter tube to cause the fluid to be delivered to the uterine cavity. In some examples, expelling the fluid from the container causes the fluid to be communicated along the lumen and out the fluid delivery port of the sonohysterogram catheter device, for delivery to the uterine cavity.

According to a further example embodiment, a sonohysterogram catheter device is disclosed that includes a catheter tube defining a lumen and a shape-retentive annular sealing member surrounding a portion of the catheter tube. The catheter tube and sealing member are cooperatively configured such that when a fluid delivery end of the catheter tube is positioned within a cervical canal of a female reproductive system, an exterior surface of the sealing member contacts tissue surrounding the cervical canal to occlude the cervical canal, with effect that fluid within a uterine cavity of the female reproductive system is contained in the uterine cavity and does not leak through the cervical canal. In some examples, the catheter tube and sealing member are cooperatively configured such that the fluid delivery end will be positioned within the cervical canal when the exterior surface of the sealing member contacts tissue surrounding the cervical canal to occlude the cervical canal.

In some examples, a front end of the sealing member is tapered with respect to a back end of the sealing member, and the fluid delivery end of the catheter tube comprises a tip that extends forward from a front end of the sealing member to form a fluid delivery port.

In some examples, the catheter devices is provided in combination with a container for storing a volume of fluid, the container being coupled to a fluid delivery port of the catheter tube. In some examples, the container is a syringe and the fluid is a normal saline or a contrast media.

In some examples, the catheter tube is semi-rigid or rigid and the sealing member comprises a medical-grade silicone or a medical-grade polymer.

In some examples, the catheter tube and sealing member are molded as a unitary rigid or semi-rigid structure from a polymer material.

In some examples, the catheter device also includes an elongated tubular housing, wherein: the sealing member is secured to a first end of the elongated tubular housing; the catheter tube insert is disposed within an annular region of the elongated tubular housing; and a handle is disposed on an outer surface of the elongated tubular housing.

### BRIEF DESCRIPTION OF THE DRAWINGS

Reference will now be made, by way of example, to the accompanying drawings which show example embodiments of the present application, and in which:

FIG. 1 an illustrative example of a female reproductive system in which the methods and devices described herein can be applied;

FIG. 2 shows a perspective view of an exemplary embodiment of a sonohysterogram catheter device, in accordance with examples of the present disclosure;

FIG. 3 shows a cross-sectional view of an exemplary embodiment of the sonohysterogram catheter device of FIG. 2, in accordance with examples of the present disclosure;

FIG. 4 shows an illustrative example of an exemplary embodiment of the sonohysterogram catheter device of FIG. 2 installed in a female reproductive system, in accordance with examples of the present disclosure;

FIG. 5 is a perspective view a further example embodiment of a sonohysterogram catheter device, in accordance with examples of the present disclosure.

FIG. 6 is a front view of the catheter tube of FIG. 5;

FIG. 7 is a sectional view of the catheter tube of FIG. 5, taken along the line VII-VII of FIG. 6;

FIG. 8 is an enlarged view of a portion of the catheter tube of FIG. 5, showing the member in greater detail;

FIG. 9 shows a sectional view of a further example embodiment of a sonohysterogram catheter device, in accordance with examples of the present disclosure.

FIG. 10 shows a further sectional view of the device of FIG. 9, in accordance with examples of the present disclosure.

FIG. 11 show sectional views illustrating further example embodiments of a sonohysterogram catheter device, in accordance with examples of the present disclosure.

FIG. 12 shows further sectional views of the devices of FIG. 11, in accordance with examples of the present disclosure.

Similar reference numerals may have been used in different figures to denote similar components.

### DETAILED DESCRIPTION

The following describes example technical solutions of this disclosure with reference to accompanying drawings. Similar reference numerals may have been used in different figures to denote similar components.

To assist in understanding the present disclosure, some relevant terminology that may be related to examples disclosed herein are introduced. In the present disclosure, the “female reproductive system” or “uterine reproductive system” can refer to the physiological system supporting fertility and reproduction, including organs such as the uterus, ovaries, fallopian tubes, vagina and cervix.

In the present disclosure, a “sonohysterogram” or a “hysterosonogram” can mean: an ultrasound procedure for observing the internal organs of the female reproductive system, including the uterus and/or the fallopian tubes. A speculum is used to allow access to, and visualization of the cervix and a narrow tip of a catheter is placed into the cervical canal. The speculum is then removed from the vagina and a transvaginal ultrasound probe is positioned in the vagina for imaging the uterine cavity and/or Fallopian tubes. A small volume of normal saline and/or contrast media may be slowly injected into the uterus via the catheter, for improving the imaging of features of the uterine cavity and/or the fallopian tubes, for example, for diagnosing abnormalities such as cervical or endometrial polyps, uterine fibroids, Mullerian uterine anomalies or for evaluating the patency of Fallopian tubes, among other possibilities.

In the present disclosure, “overmolding” can mean: an injection molding technique for manufacturing a single component comprising two or more materials, for example, comprising a plastic (e.g., thermoplastic) molded over a substrate.

As used herein “proximal end”, “front end”, “distal end”, and “back end”, when referring to components of a catheter device, are relative terms, wherein the terms “proximal end” and “front end” of the component each refer to the end of the component that is closest to a fluid delivery end of the catheter device (i.e., the end of the catheter device that introduces fluid into the female reproductive system), and the terms “distal end” and “back end" of the component each refer to the end of the component that is farthest from the fluid dispending end of the catheter device.

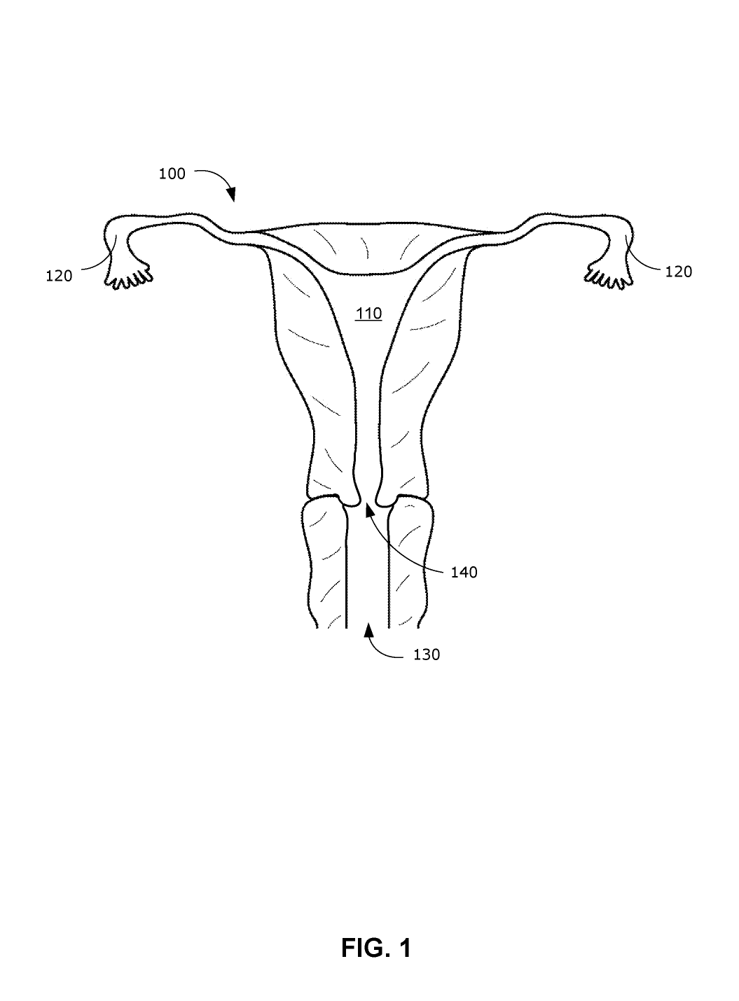

FIG. 1 shows an illustrative example of a female reproductive system 100 in which the methods and devices described herein can be applied, in accordance with examples of the present disclosure. The system 100 has been simplified in this example for ease of understanding. For exemplary purposes, internal organs of the female reproductive system 100 shown in FIG. 1 include the uterus 110, fallopian tubes 120, the vagina (or vaginal tract) 130 and the cervix (or cervical canal) 140. In the present disclosure, the terms “cervix” and “cervical canal” may be used interchangeably. In examples, the cervix 140 may further include an internal OS representing the upper boundary of the cervix 140 (e.g., between the cervix 140 and the uterus 110), and an external OS representing the lower boundary of the cervix 140 (e.g., between the cervix 140 and the vagina 130).

In examples, during a sonohysterogram procedure, a fluid such as normal saline and/or contrast media (e.g., ExEm® Foam, Echovist®), may be introduced into the uterus 110 through the cervix 140, for example, to assist in ultrasound imaging of the uterus 110 and/or fallopian tubes 120, among other internal organs of the female reproductive system 100. In examples, following the insertion of the fluid into the uterus 110, a sonohysterogram procedure may capture ultrasound images of the uterus 110 and/or fallopian tubes 120 using a transvaginal ultrasound probe. In some embodiments, for example, the presence of a small volume of fluid in the uterus during the sonohysterogram procedure may allow the ultrasound imaging to capture structures of the uterus 110, fallopian tubes 120 and/or cervical canal 140, among other internal organs of the female reproductive system 100, in greater detail than a standard transvaginal ultrasound performed without the addition of fluid.

In examples, a traditional sonogram catheter may deliver the fluid into the uterus 110 through a thin flexible tube (e.g., catheter) coupled to a syringe at one end and having a small, concentric balloon that is inflated inside the uterus 100, after placement of the catheter through the cervical opening. In examples, inflation of a soft, concentric balloon above the internal OS enables occlusion of the cervical opening, for example, for preventing leakage of the fluid. However, such sonogram catheters can be expensive, particularly for clinics performing many sonohysterogram procedures daily, and can hinder the ability of an operator to obtain clear ultrasound images, for example, as the inflated balloon may obscure portions of the uterine cavity. Furthermore, traditional sonogram catheters are known to cause discomfort to patients (particularly due to the inflated balloon) during sonohysterogram procedures.

The present disclosure describes examples that may help to address some or all of the above drawbacks of existing technologies.

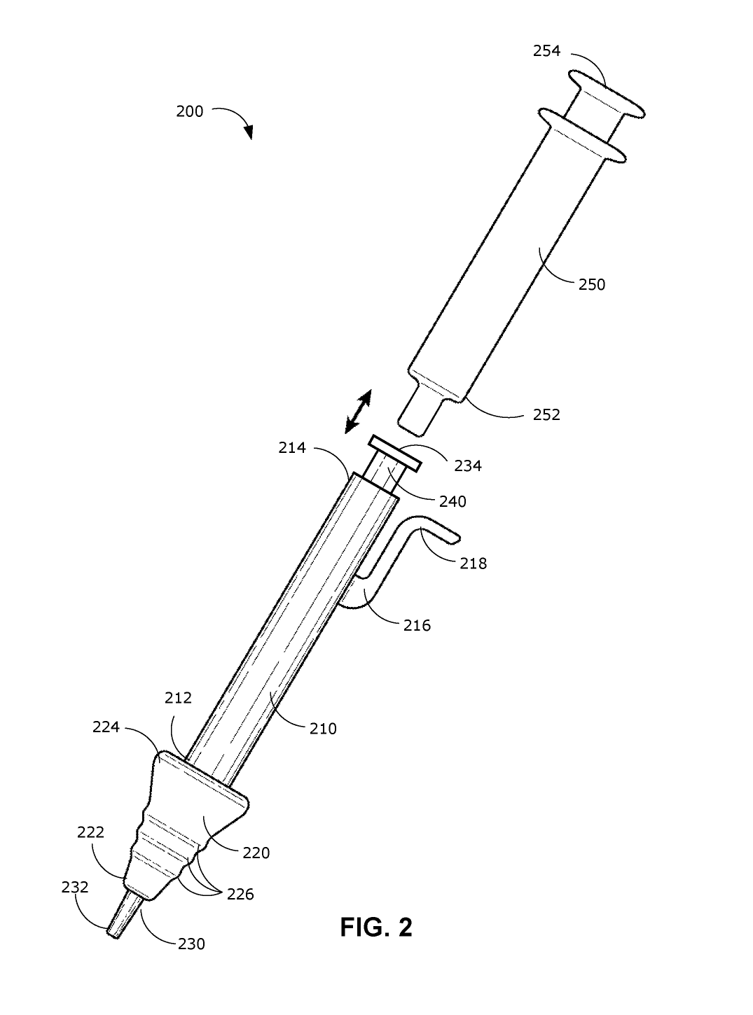

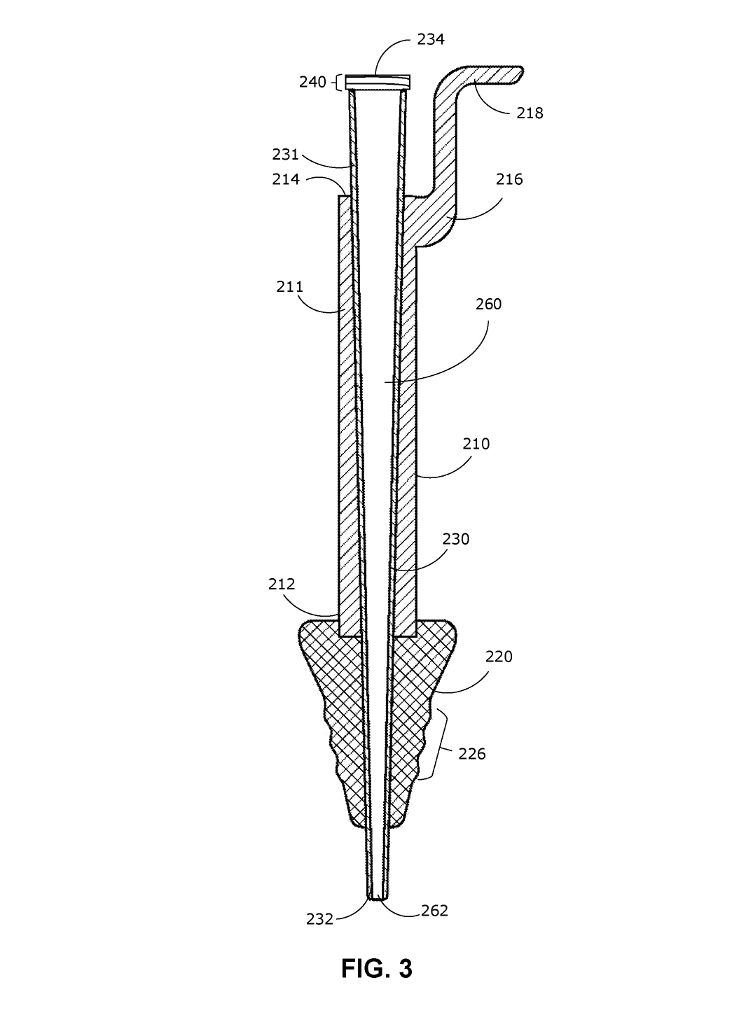

FIG. 2 shows an exemplary embodiment of a sonohysterogram catheter device 200, in accordance with examples of the present disclosure. In examples, the sonohysterogram catheter device 200 may include a catheter housing 210, a sealing member 220 (also referred to as a cervical occupier), catheter insert 230 (also referred to herein as a catheter tube 230), and a locking element 240. In example embodiments, the sonohysterogram catheter device 200 is configured to be used with a fluid-holding container 250 (for example, a syringe), where the container 250 is configured to be secured to a distal end (e.g., fluid receiving end) of catheter tube 230 via the locking element 240. As best seen in FIG. 3, in the illustrated example, the catheter tube 230 has an elongated tubular body that defines a lumen 260 that provides a fluid path between a fluid receiving port 234 at a fluid receiving end of the catheter tube 230 and a fluid delivery port 262 at a fluid delivery end of the catheter tube 230.

In some embodiments, for example, the catheter housing 210 may be an elongated, tubular shaped housing extending between a first end 212 (e.g., a front end) and a second end 214 (a back end). In examples, the catheter housing 210 may be rigid, for example, produced from a rigid medical-grade polymer (e.g., polyethylene, polyvinyl chloride, among other possibilities) and may include an exterior surface and an interior lumen. In examples, the catheter housing 210 may include at least one handle 216 projecting outwardly from the exterior surface of the catheter housing 210. Although FIG. 2 shows a single handle 216, it is understood that additional handles may be included for assisting users in holding the sonohysterogram catheter device 200 during use. In some embodiments, for example, the handle 216 may extend laterally toward the second end 214 before curving and extending outwardly, for example, forming a flange 218, among other possibilities, for receiving a respective one or more fingers of a user holding the sonohysterogram catheter device 200.

In examples, the sealing member 220 may be coupled to the first end 212 of the catheter housing 210. In some embodiments, the sealing member 220 may be tapered, for example, having a proximal end 222 (e.g., front end) that is smaller in diameter than a distal end 224 (e.g., back end). In examples, an exterior surface of the sealing member 220 may be substantially smooth, for example, including beveled edges at the proximal end 222 and the distal end 224 for minimizing discomfort during use and assisting in placement of the device 200 in the vaginal tract, and for ensuring a tight seal at the cervical canal 140. In some embodiments, for example, the exterior surface of the sealing member 220 may include one or more concentric ridges 226 or textured elements, for example, disposed around a circumference of the outer surface of the sealing member 220 for improving a seal formed between the sealing member 220 and the cervical canal 140, thereby further preventing leakage or backflow of fluid through the cervical canal 140. In some embodiments, for example, the sealing member 220 may be semi-rigid or rigid, for example, comprising medical-grade silicone or another biocompatible material, such as a medical-grade polymer, among other possibilities. In examples, the sealing member 220 further includes an annulus or lumen extending axially through the sealing member 220, for example, forming a first through-hole at the proximal end 222 and extending to form a second through-hole at the distal end 224, for example, as shown with respect to FIG. 3.

In some embodiments, for example, the catheter tube 230 may be a semi-rigid or rigid tube, for example, tapering to a narrow tip 232 at one end and forming a fluid receiving port 234 at an opposite end. The narrow tip 232 defines the fluid delivery port 262 that is in fluid communication with the fluid receiving port 234 via lumen 260. In examples, the fluid receiving port 234 may include the locking element 240, for example, for coupling with an end 252 of the container 250. For example, as shown in FIG. 3, the locking element 240 may be disposed on an outer surface of the catheter tube 230 and may include a luer lock or similar threaded locking mechanism configured to engage with the end 252 of the container 250. In some embodiments, for example, the locking element 240 may represent a small groove on an outer surface of the catheter tube 230, wherein the end 252 of the container 250 is received into the fluid receiving port 234 and the container 250 is secured to the catheter tube 230, for example, via a rotation of the container 250 (e.g., a screwing-in motion) by the user to engage with the locking element 240. In other embodiments, the end 252 of the syringe 250 may represent a threaded tip and the locking element 240 may include a luer lock or similar threaded locking mechanism configured to engage with the threaded tip of the syringe 250, among other possibilities.

Following installation of the container 250 to the catheter tube 230, fluid may be received into the fluid receiving port 234, (e.g., fluid contained within the container 250). In some embodiments, for example, the container 250 may be a syringe, and the fluid may be received into the fluid receiving port 234, for example, responsive to a user depressing a plunger 254 of the syringe.

In examples, the catheter tube 230 may be disposed within the respective lumens of the catheter housing 210 and the sealing member 220, for example, where the tip 232 may protrude out of the first through-hole of the sealing member 220. In some embodiments, for example, when the catheter tube 230 is disposed within the catheter housing 210 and the sealing member 220, a portion of the catheter tube 230 including the fluid receiving port 234 may extend beyond the second end 214 of the catheter housing 210, for example, as shown in FIG. 2. In other embodiments, for example, the portion of the catheter tube 230 including the fluid receiving port 234 may not extend beyond the second end 214 of the catheter housing 210, among other possibilities.

FIG. 3 shows a cross-sectional view of an exemplary embodiment of the sonohysterogram catheter device 200, in accordance with examples of the present disclosure. In examples, the catheter tube 230 is shown disposed within a progressively tapered inner lumen of the catheter housing 210. In examples, a thickness of a wall 231 of the catheter tube 230 is shown as being consistent along the length of the catheter tube 230, whereas the diameter of the catheter tube 230 progressively narrows along the length of the catheter tube from the second end 214 to the first end 212.

In some embodiments, for example, a thickness of a wall 211 of the catheter housing 210 may be thinner at the second end 214 than at the first end 212, for example, where the progressively thicker wall 211 defines the progressively tapered inner lumen of the catheter housing 210, however other configurations may be used. For example, a thickness of wall 211 may be consistent along the length of the catheter housing 210, where the diameter of the catheter housing 210 may progressively narrow along the length of the catheter housing 210 to accommodate the catheter tube 230, among other possibilities.

In some embodiments, for example, the sonohysterogram catheter device 200 may be assembled by securely fitting the sealing member 220 over the first end 212 of the catheter housing 210, for example, to form a friction fit and/or may be fixed in place using a medical-grade epoxy or glue, among other possibilities. In other embodiments, for example, the sonohysterogram catheter device 200 may be assembled by injection overmolding, for example, where the sealing member 220 may be overmolded directly on to the catheter housing 210 (e.g., at the first end 212), for example, bonding the sealing member 220 directly to a pre-determined surface area of the first end 212 of the catheter housing 210, among other possibilities.

In examples, as shown in FIG. 3, the interior lumen of the catheter housing 210 may be coincident with the interior lumen of the sealing member 220, for example, for receiving the catheter tube 230. In examples, the catheter tube 230 may be coupled to the catheter housing 210 and sealing cap 220 by sliding the tip 232 end of the catheter tube 230 into the second end 214 of the catheter housing 210, where a depth of insertion into the catheter housing 210 and sealing member 220 is limited by the diameter of the first through-hole and an outer diameter of the catheter tube 230. For example, the tip 232 may maximally protrude from the first through-hole of the sealing member 220 based on a friction fit between the first through-hole and the outer diameter of the catheter tube 230.

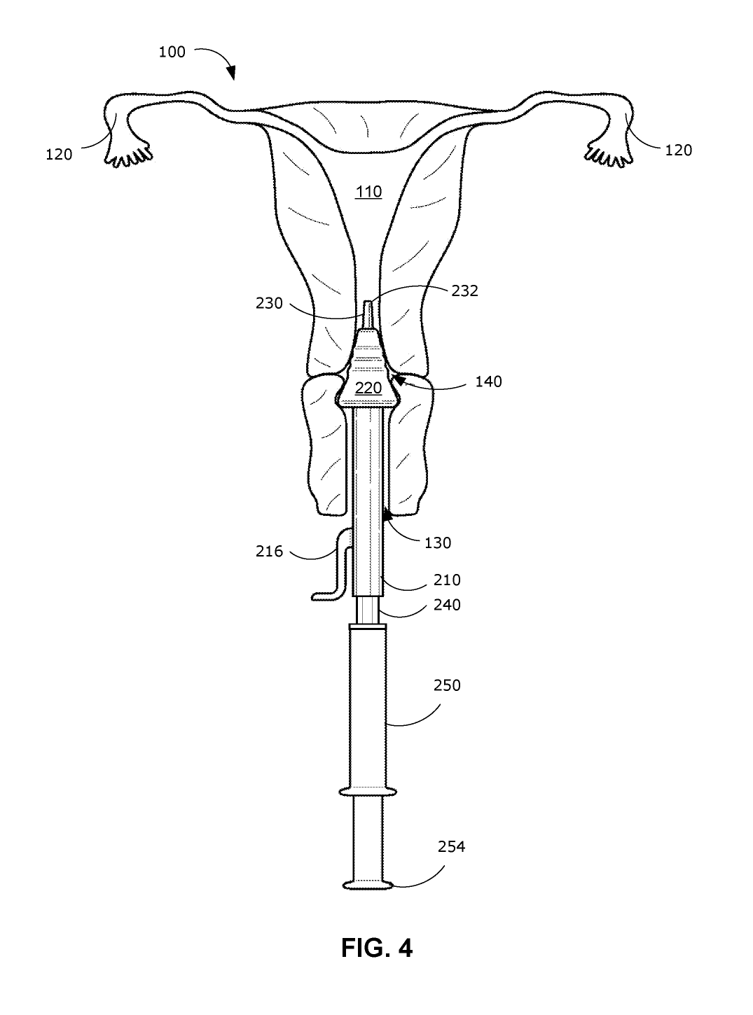

FIG. 4 shows an illustrative example of an exemplary embodiment of the sonohysterogram catheter device 200 positioned in a female reproductive system 100 as part of a diagnostic procedure, in accordance with examples of the present disclosure. In examples, the sonohysterogram catheter device 200 may be inserted along the vaginal tract 130 and positioned at the cervix 140 of the female reproductive system 100, for example, where the outer surface of the sealing member 220 abuts the external OS of the cervix 140 (e.g., subject to an applied abutment pressure in an axial direction by the user) and the tip 232 enters the cervical canal 140, but not the uterus 110, for example, prior to and during a sonohysterogram procedure, after which the sonohysterogram catheter device 200 may be removed from the vaginal and cervical region. In examples, a depth of insertion of the catheter tip into the cervical canal 140 may be limited by the diameter of the cervix 140 and/or cervical opening (e.g., external OS) in contact with the external surface of the sealing member 220, such that the tip 232 is located in the cervical canal 140 without extending into the uterus 110 and the sealing member 220 in contact with the cervix 140 forms a fluid-retentive seal preventing the leakage or flow of fluid contained in the uterus 110, for example, flowing back from the uterus 110 through the cervix 140.

In examples, the sonohysterogram catheter device 200 may be configured to be in fluid communication with the uterus 110 of the female reproductive system 100, for example, for delivering a fluid such as normal saline and/or a contrast media, among other possibilities, into the uterus 110. For example, when the container 250 (e.g., syringe) is coupled to the catheter tube 230 via the locking mechanism 240, and responsive to an action such as a user depressing the plunger 254 of the syringe, fluid contained within the container 250 may be expelled out of the end 252 of the container 250 and received into the fluid receiving port 234, where it may be communicated along the interior lumen of the catheter tube 230 and out the tip 232, for example, for providing the fluid to the uterus 110. In examples, operation of the sonohysterogram catheter device 200 may be performed by a user grasping the handle 216 with one or more fingers of one hand and depressing the plunger 245 of the syringe, for example, using the thumb of another hand, among other possibilities.

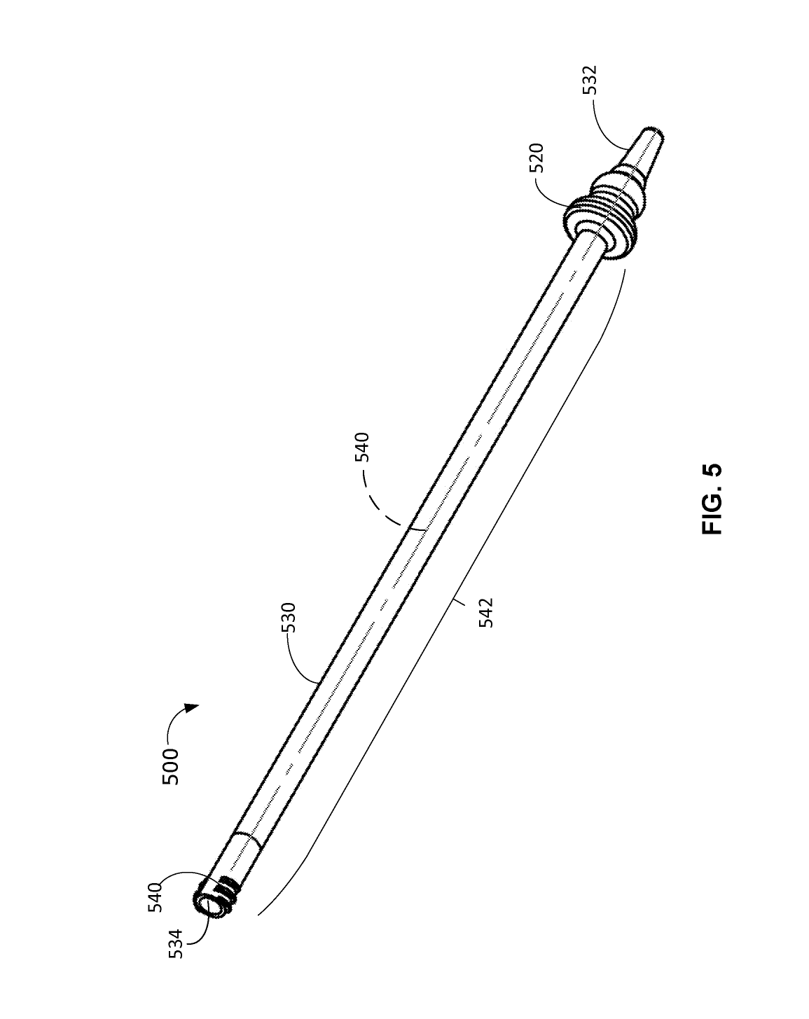

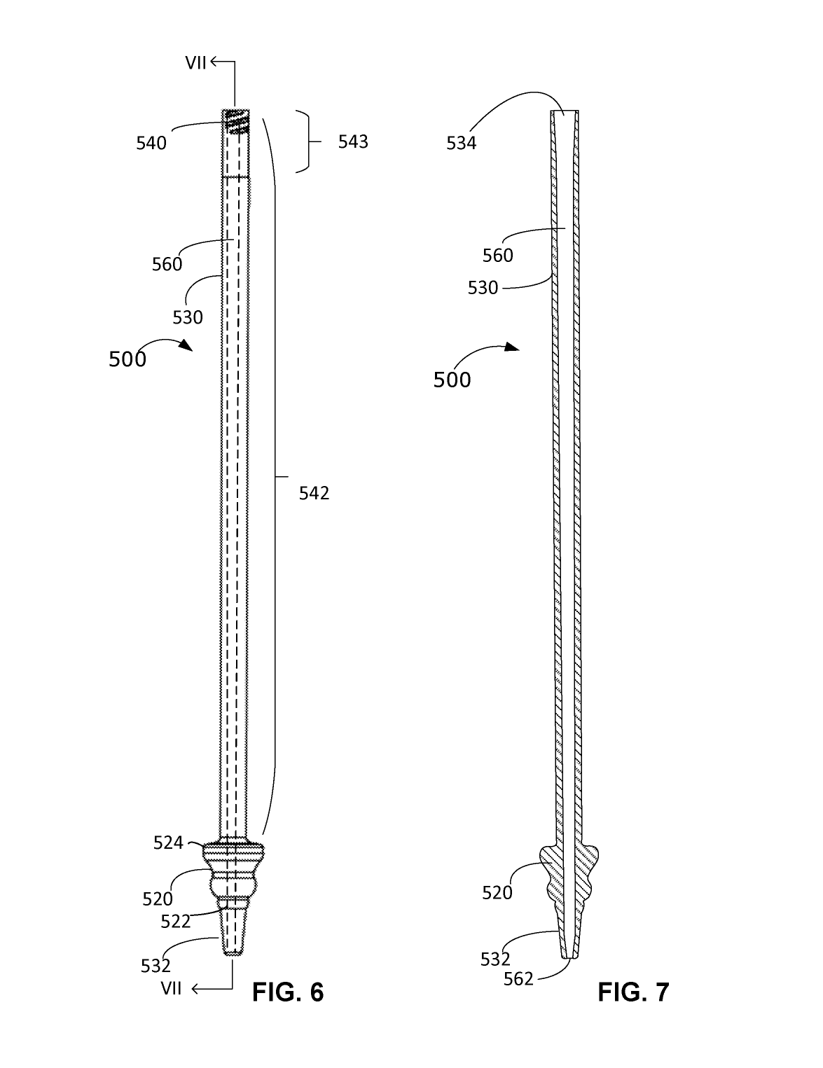

In a further example embodiment, a sonohysterogram catheter device is disclosed that is similar to device 200 but does not have an external housing and has a sealing member formed integrally with the catheter tube. In this regard, FIGS. 5 to 8 illustrate a further example of a sonohysterogram catheter device 500 according to a further example embodiment.

As shown in FIG. 5 to 7, the sonohysterogram catheter device 500 includes a catheter tube 530 that is formed by an elongated, tubular body potion 542, an annular sealing member 520 and a tip 532 that collectively define a lumen 560 that extends from a fluid receiving port 534 located at an fluid receiving end of the catheter tube 530 to a delivery port 262 located at a fluid delivery end of the catheter tube 530. In example implementations, catheter tube 530 is molded as a complete unit from a single material such that elongate tubular body portion 542, sealing member 520 and tip 532 are all part the same unitary molded structure. In one example, the catheter tube 530 is formed from a polymer material, for example a thermoplastic polyurethane (TPU) material, that enables the catheter tube 530 to have sufficient elasticity, flexibility and rigidity to be used for its intended purpose, and that is formulated to meet medical use standards such as ISO 10993 and USP Class VI standards. In examples, the catheter tube 530 is formed from a flexible TPU based material that is configured to have temporary shape retention characteristics such that the elongate tubular body portion 542 can be bent to form a curve along its longitudinal axis 540 to facilitate use of the catheter tube 530 and will retain that curve at least temporarily for a time period corresponding to a typical use time of a sonohysterogram catheter. In some examples, the polymer material used for the sonohysterogram catheter device 500 has a Shore hardness within a Shore hardness range of 50 to 70 Shore D. Alternative polymer materials suitable for forming catheter tube 530 in may include, but are not limited to: polyethylene (PE); polytetrafluoroethylene (PTFE); silicone; or polyvinyl chloride (PVC).

The elongate tubular body portion 542 of the catheter tube 530 defines the fluid receiving port 534 that is located at the fluid receiving end of the catheter tube 542. In the illustrated example, the catheter tube 530 includes a locking element 540 for securing fluid container 250 to the catheter tube 530 such that a fluid can be transferred from the fluid container 250 into lumen 560 via the fluid receiving port 534. In some examples the locking element 540 is configured to mate with a Luer lock tip of a syringe and includes one or more thread components that protrude radially from the elongate tubular body portion 542 near the fluid receiving end. In some examples, the back end region 543 of the elongate tubular body portion 542 where the locking element 540 is located and fluid receiving port 534 is defined could be formed from a polymer material that is harder and more rigid than that used for the remainder of the catheter tube 530.

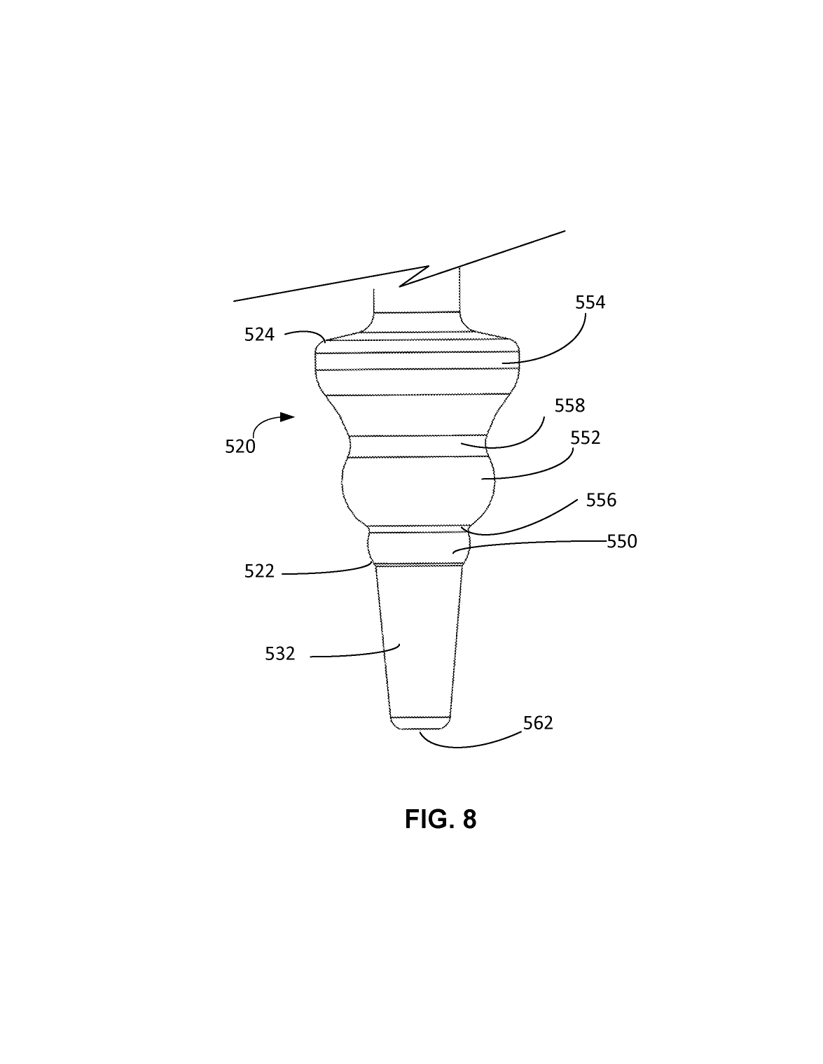

Sealing member 520 is located at the front end of the elongate tubular body portion 542. Similar to the sealing member 220 described above, the sealing member 520 also has an outer profile that is tapering such that the proximal or forward end 522 of the sealing member 520 is smaller in diameter than its distal or back end 524. In the illustrated example, as best seen in FIG. 8, the exterior surface of the sealing member 520 includes a plurality of concentric ridges 556, 552, 554 and intervening groves 556, 558, disposed around an outer circumference of the sealing member 520 for improving a seal formed between the sealing member 220 and the cervical canal 140, thereby further preventing leakage or backflow of fluid through the cervical canal 140. In the illustrated examples, the respective outer diameters of concentric ridges 550, 552 and 554 and their intervening groves 556, 558 increase in outer diameter size the further the ridges are from the fluid delivery port 562. The plurality of increasingly sized concentric ridges 556, 552, 554 and intervening groves 556, 558 enable the sealing member to sealingly engage different sized cervical external OSs.

Tip 532, which also tapers in a forward direction, extends from the forward end 522 of the sealing member 520 and defines the fluid delivery port 562.

Sonohysterogram catheter device 500 can be used in a similar manner as is illustrated in FIG. 4 in respect of sonohysterogram catheter device 200. In this regard, catheter device 500 can be positioned in a female reproductive system 100 as follows. In examples, the catheter tube 500 may be inserted along the vaginal tract 130 and positioned at the cervix 140 of the female reproductive system 100, for example, where the outer surface of the sealing member 520 abuts the external OS of the cervix 140 (e.g., subject to an applied abutment pressure in an axial direction by the user) and the tip 532 enters the cervical canal 140, for example, prior to and during a sonohysterogram procedure, after which the sonohysterogram catheter device 500 may be removed from the vaginal and cervical region. In examples, a depth of insertion of the catheter tip 532 into the cervical canal 140 may be limited by the diameter of the cervix 140 and/or cervical opening (e.g., external OS) in contact with the external surface of the sealing member 520, and where the sealing member 520 in contact with external OS of the cervix 140 forms a fluid-retentive seal preventing the leakage or flow of fluid contained in the uterus 110, for example, flowing back from the uterus 110 through the cervix 140. It will be appreciated that the annular sealing member 520 is located proximate the fluid delivery end of the catheter tube such that when the fluid delivery end (e.g., end of tip 532) of the catheter tube is positioned within a cervical canal of a female reproductive system, an exterior surface of the sealing member contacts tissue surrounding the cervical canal to occlude the cervical canal and from a barrier to leakage of fluid, with effect that fluid delivered to a uterine cavity of the female reproductive system is contained in the uterine cavity and is prevented from escaping through the cervical canal.

In example embodiments, the dimensions of the sonohysterogram catheter device 500 are configured such that the elongate tubular body portion 542 is a sufficient length to extend through the vaginal tract 130 such that the sealing member 520 can sealing engage the external OS of the cervix 140 with the tip 532 protruding into the endocervical canal while permitting the fluid-holding container 250 to be located externally of the vaginal tract 130. The sealing member 520 and tip 532 are configured such that when the sealing member 520 engages the cervix 140 to form a leakage barrier, the front end of the tip 532 is located within the cervical canal without protruding into the uterus 110.

For illustrative purposes, example dimensions of one possible configuration of catheter device 500 are provided as follows. It will be noted that the following dimensions are not restrictive, and different configurations can be used that have different inventions. In an illustrative example: the sonohysterogram catheter device 500 has an overall length from fluid receiving port 534 to fluid delivery port 562 that is within a range of 200mm to 240mm, for example 220mm; elongate tubular body portion 542 has a length that is within the range of 170mm to 210mm, for example 190mm, and has a substantially constant outer diameter along its entire length (e.g., an outer diameter within the range of 5mm to 10mm, for example 6.3mm); the sealing member 520 has a length within the range of 10mm and 30mm, for example 18.5mm, with the ridge 554 having an outer diameter within the range of 10mm to 20mm, for example 15mm, ridge 552 having an outer diameter less than that of ridge 554 and within the range of 8mm to 12mm, for example 10mm, ridge 550 having an outer diameter less than that of ridge 552 and within the range of 6mm to 8mm, for example 7mm, groove 558 having an outer diameter less than the outer diameters of each of the ridges 554 and 552, and groove 556 having an outer diameter less than the outer diameters of each of ridges 552 and 550; the tip 532 has a length of between 12mm and 20mm, for example 12mm, with an outer diameter that tapers inwardly from its back end (i.e. where it joins sealing member 520) towards its front end (i.e., where it defines the fluid delivery port 562); in one example, the back end of the tip 532 has an outer diameter of between 5mm and 7mm, for example 6.4mm and the front end of tip 532 has an outer diameter of between 4mm and 6mm, for example 4.4mm.

With reference to FIG. 7, in the illustrated embodiment, the fluid receiving port 534 and fluid delivery port 562 are axially aligned and located at opposite ends of lumen 560. In one example, the lumen 562 tapers along the length of the catheter device 500 from a diameter of between 3mm and 8mm, for example 4.8mm, at fluid receiving port 534 to a diameter of between 1.5mm and 4mm, for example 2mm at the fluid delivery port 562. In some examples the angle of the taper of lumen 560 can vary along the length of the lumen as shown in FIG. 7.

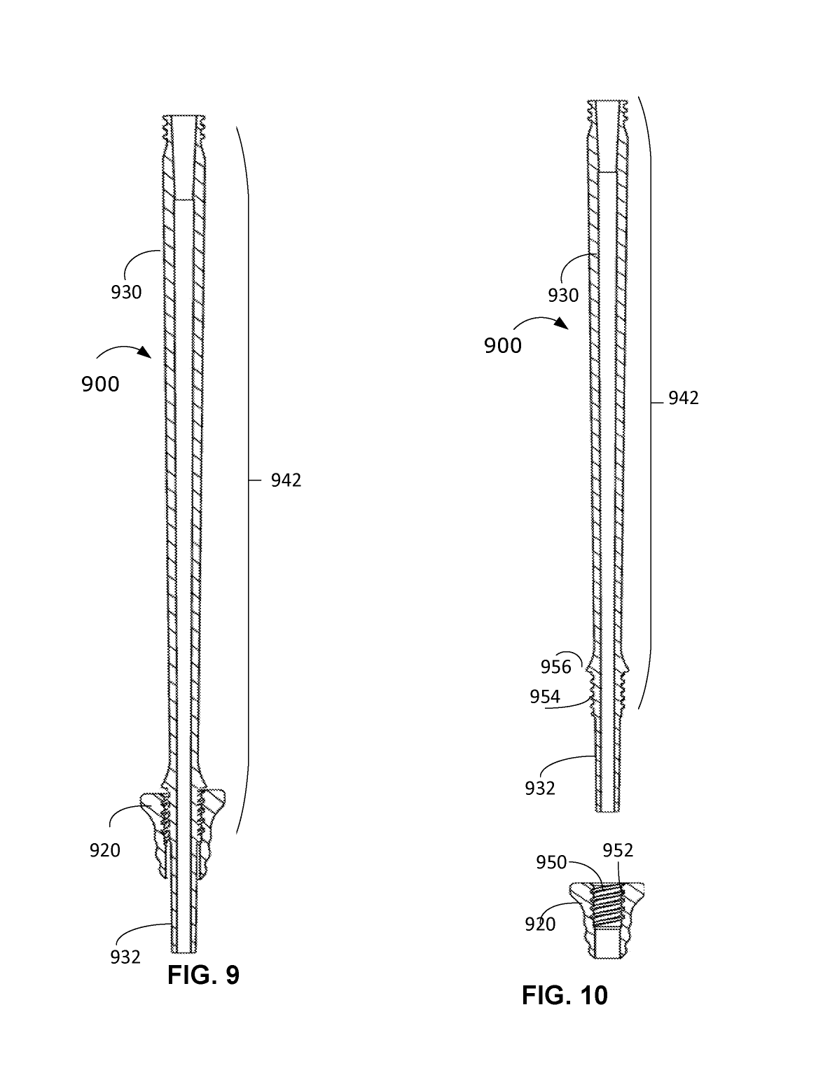

With reference to FIGS. 9 and 10, a version of a sonohysterogram catheter device 900 is disclosed in accordance with a further example embodiment that is generally similar to sonohysterogram catheter device 500 described above except that, in the case of sonohysterogram catheter device 900, the annular sealing member 920 is not integrally formed with the remainder of the catheter tube 930, but rather is a separate component that can be mounted to the elongate tubular body portion 942 of the catheter tube 930. FIG. 9 shows the annular sealing member 920 mounted to the remainder of the catheter tube 930, and FIG. 10 shows the annular sealing member 920 detached from the remainder of the catheter tube 930. The catheter device 900, the elongate tubular body portion 942 and tip 932 are molded as a unitary structure and the annular sealing member 920 is molded independently. In some examples, the elongate tubular body portion 942 and tip 932 are formed from a first polymer material (such as one of the materials noted above), and the annular sealing member 920 is molded from the same material. In some examples, the elongate tubular body portion 942 and tip 932 are formed from a first polymer material (such as one of the materials noted above), and the annular sealing member 920 is molded from a different material ( for example, such as a different one of the materials listed above) that may, for example, be a harder polymer material, or alternatively, a softer polymer material than the first polymer material.

The use of a mountable annular sealing member 920 enables the catheter tube 930 to be configured with different configurations and sizes of annular sealing members.

In at least some examples, the mounted position of the sealing member 920 along the length of the elongate tubular portion 942 is adjustable, thereby allowing the distance that the front end (i.e. fluid delivery end) of tip 932 protrudes forward of the front end of the sealing member 920 to be adjustable by a user. In one example, this distance can be adjustable to be within a range 10mm to 20mm, thereby allowing the tip distance to be adjusted to accommodate different anatomically sized female reproductive systems. In one example, the sealing member 920 includes a central opening 950 that is sized to slide over the elongate tubular body portion 942. The central opening 950 includes a threaded region 952 on its inner surface for engaging with a corresponding threaded region 954 that is provide on an outer circumference of the elongate tubular body portion 942. The extent to which the threaded region 952 of the sealing member 920 is screwed onto the threaded region 954 of the elongate tubular body portion 942 determines the distance that the tip 932 extends from the front end of the sealing member 920. A stop limit structure 956 can be provided on the tubular body portion 942 at a back end of the threaded region 954 to limit the distance that the sealing member 920 can be screwed onto the tubular body portion 942. As seen in FIG. 9, the elongate tubular body portion 942 can include a slight taper along its length, getting narrower from its back end until a region proximate the stop limit structure 956.

Accordingly, in the example of FIGS. 9 and 10, the catheter tube 930 includes an elongated tubular body portion 942 and a tip 932 collectively defining the lumen, wherein the tip 932 has a forward end that defines the fluid delivery end of the catheter tube, the annular sealing member 920 being mountable to the elongated tubular body portion 942. The annular sealing member 930 and elongated tubular body portion 942 comprise cooperating threaded regions 952, 954 for mounting the annular sealing member 920 to the elongated tubular body portion 942, wherein a distance that the forward end of the fluid delivery port extends forward of a forward end of the sealing member is adjustable.

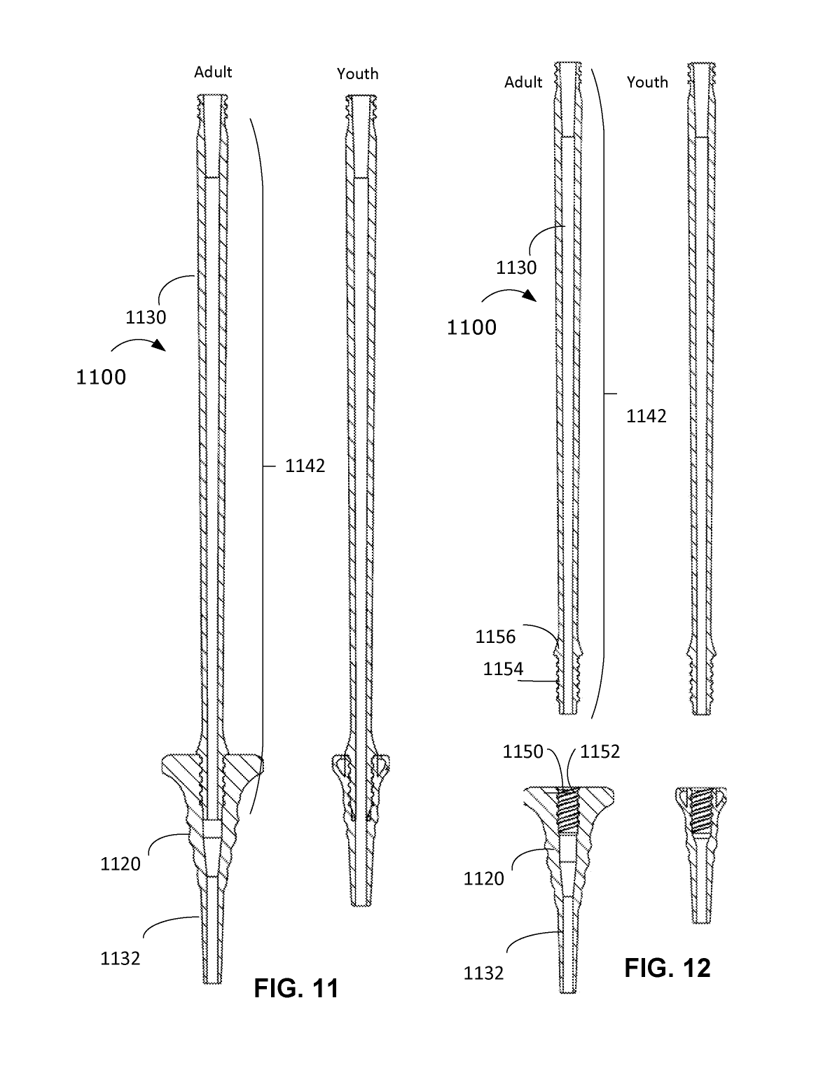

With reference to FIGS. 11 and 12, a further version of a sonohysterogram catheter device 1100 is disclosed in accordance with a further example embodiment that is generally similar to sonohysterogram catheter device 900 described above except that, in the case of sonohysterogram catheter device 1100, the annular sealing member 1120 and tip 1132 are integrally formed with each other. The back end of sealing member 1120 includes a central opening 1150 with an internal threaded region 1152 for securing to a threaded region 1154 that is provided on an outer circumference of a lower end of the elongate tubular body portion 1142. A stop limit structure 1156 can be provided on the tubular body portion 1142 at a back end of the threaded region 1154 to limit the distance that the sealing member 920 can be screwed onto the tubular body portion 1142. FIG. 11 shows sonohysterogram catheter device 1100 with two different configurations (e.g. an adult version and a smaller youth version) of annular sealing member 1120 with integrated tip 1132 being mounted to the device. FIG. 12 shows the devices 1100 with annular sealing members 1120 with integrated tip 1132 being unmounted.

Thus, in the embodiments of FOG. 11 and 12, the catheter tube 1130 comprises an elongated tubular body portion 1142 having a threaded region 1154 at a font end portion thereof. A tip 1132 extends from a front end of the sealing member 1120 to define the fluid delivery end of the catheter tube 1130. The annular sealing member 1120 and the tip 1132 form a unity structure with a threaded region 1142 located at a back end of a lumen portion that passes through the annular sealing member 1120. The sealing member 1120 can be mounted to the elongated tubular body portion 1142 by screwing the threaded region 1152 of the annular sealing member onto the threaded region 1154 of the elongated tubular body portion 1142. When the sealing member 1120 is mounted to the elongated tubular body portion 1142, the elongated tubular body portion1142, the annular sealing member 1120 and the tip collectively 1120 define the lumen of the catheter tube 1130.

The use of a mountable annular sealing member 1120 enables the catheter tube 1130 to be configured with different configurations and sizes of annular sealing members.

Although the present disclosure describes methods and processes with steps in a certain order, one or more steps of the methods and processes may be omitted or altered as appropriate. One or more steps may take place in an order other than that in which they are described, as appropriate.

The present disclosure may be embodied in other specific forms without departing from the subject matter of the claims. The described example embodiments are to be considered in all respects as being only illustrative and not restrictive. Selected features from one or more of the above-described embodiments may be combined to create alternative embodiments not explicitly described, features suitable for such combinations being understood within the scope of this disclosure.

All values and sub-ranges within disclosed ranges are also disclosed. Also, although the systems, devices and processes disclosed and shown herein may comprise a specific number of elements/components, the systems, devices and assemblies could be modified to include additional or fewer of such elements/components. For example, although any of the elements/components disclosed may be referenced as being singular, the embodiments disclosed herein could be modified to include a plurality of such elements/components. The subject matter described herein intends to cover and embrace all suitable changes in technology. As used herein, terms such as “substantially”, “generally” and “approximately” mean within a reasonable range or tolerance of the stated value, such that the variation does not materially alter the intended function or result. Unless otherwise specified, this range may include variations of ±10%, or as understood by one skilled in the art. When a range for a value is provided, the range includes the values that are stated to define the scope of the range.

1. A sonohysterogram catheter device comprising
a catheter tube defining a lumen that extends a length of the catheter tube from a fluid receiving port at a fluid receiving end of the catheter tube to a fluid delivery port at a fluid delivery end of the catheter tube, wherein an annular sealing member is located proximate the fluid delivery end of the catheter tube such that when the fluid delivery end of the catheter tube is positioned within a cervical canal of a female reproductive system, an exterior surface of the annular sealing member contacts tissue surrounding the cervical canal to occlude the cervical canal and prevent fluid from escaping a uterine cavity of the female reproductive system.

2. The sonohysterogram catheter device of claim 1
wherein the catheter tube comprises an elongated tubular body portion extending from the fluid receiving end to a back end of the annular sealing member and a tip extending from a front end of the annular sealing member to form the fluid delivery end, the elongated tubular body portion, the annular sealing member and the tip collectively defining the lumen.

3. The sonohysterogram catheter device of claim 2
wherein the annular sealing member and the tip are collectively configured such that the fluid delivery end will be positioned within the cervical canal when the exterior surface of the annular sealing member occludes the cervical canal.

4. The sonohysterogram catheter device of claim 2
wherein the elongated tubular body portion, annular sealing member and tip are molded as a unitary rigid or semi-rigid structure from a polymer material.

5. The sonohysterogram catheter device of claim 2
wherein the annular sealing member tapers generally from a larger diameter at the back end of the annular sealing member to a smaller diameter at the front end of the annular sealing member and includes a plurality of annular ridges that decrease in an outer diameter size from the back end to the front end.

6. The sonohysterogram catheter device of claim 2
wherein the outer diameter of the tip tapers from a larger outer diameter at a back end of the tip to a smaller outer diameter at the fluid delivery port.

7. The sonohysterogram catheter device of claim 2
wherein a length of the tip from the annular sealing member to the fluid delivery port is equal to or less than 20mm.

8. The sonohysterogram catheter device of claim 2
wherein the elongated tubular body portion is deformable to form a curve along a length thereof.

9. The sonohysterogram catheter device of claim 2
wherein one or more thread elements are formed on an outer circumference of the elongated tubular body proximate the fluid receiving end to enable a fluid container to be secured thereto to deliver a fluid from the fluid container to the lumen via the fluid receiving port.

10. The sonohysterogram catheter device of claim 1
wherein the catheter tube comprises an elongated tubular body portion and a tip collectively defining the lumen, wherein the tip has a forward end that defines the fluid delivery end of the catheter tube, the annular sealing member being mountable to the elongated tubular body portion.

11. The sonohysterogram catheter device of claim 10
wherein the annular sealing member and elongated tubular body portion comprise cooperating threaded regions for mounting the annular sealing member to the elongated tubular body portion, wherein a distance that the forward end of the fluid delivery port extends forward of a forward end of the sealing member is adjustable.

12. The sonohysterogram catheter device of claim 1 wherein:
the catheter tube comprises an elongated tubular body portion having a threaded region at a font end portion thereof;
a tip extends from a front end of the sealing member to define the fluid delivery end of the catheter tube, the annular sealing member and the tip form a unity structure with a threaded region located at a back end of a lumen portion that passes through the annular sealing member;
wherein the sealing member can be mounted to the elongated tubular body portion by screwing the threaded region of the annular sealing member onto the threaded region of the elongated tubular body portion; and
wherein when the sealing member is mounted to the elongated tubular body portion, the elongated tubular body portion, the annular sealing member and the tip collectively defined the lumen.

13. A method for delivering fluid into a uterine cavity of a female reproductive system, the method comprising:
positioning the catheter tube of the sonohysterogram catheter device of claim 1 along a vaginal tract of the female reproductive system and in abutment with the tissue surrounding the cervical canal of the female reproductive system;
applying an abutment pressure to the sonohysterogram catheter device for providing a fluid seal between the sealing member and the tissue surrounding the cervical canal; and
expelling the fluid from a container secured to the fluid receiving port at the fluid receiving end of the catheter tube to cause the fluid to be delivered to the uterine cavity.

14. The method of claim 13, wherein expelling the fluid from the container causes the fluid to be communicated along the lumen and out the fluid delivery port of the sonohysterogram catheter device, for delivery to the uterine cavity.

15. The method of claim 13, wherein expelling the fluid from the container comprises:
depressing a plunger of the container to cause the fluid to be expelled out an end of the container and into the fluid receiving port.

16. A sonohysterogram catheter device comprising:
a catheter tube defining a lumen; and
a shape-retentive annular sealing member surrounding a portion of the catheter tube;
the catheter tube and sealing member being cooperatively configured such that when a fluid delivery end of the catheter tube is positioned within a cervical canal of a female reproductive system, an exterior surface of the sealing member contacts tissue surrounding the cervical canal to occlude the cervical canal, with effect that fluid within a uterine cavity of the female reproductive system is contained in the uterine cavity and does not leak through the cervical canal.

17. The catheter device of claim 16, wherein a front end of the sealing member is tapered with respect to a back end of the sealing member, and the fluid delivery end of the catheter tube comprises a tip that extends forward from a front end of the sealing member to form a fluid delivery port.

18. The catheter device of claim 17, in combination with a container for storing a volume of fluid, the container being coupled to a fluid delivery port of the catheter tube, wherein the container comprises a syringe and the fluid is a normal saline or a contrast media.

19. The catheter device of claim 16 wherein the catheter tube and sealing member are molded as a unitary rigid or semi-rigid structure from a polymer material.

20. The catheter device of claim 16 wherein the catheter tube and sealing member are cooperatively configured such that the fluid delivery end will be positioned within the cervical canal when the exterior surface of the sealing member contacts tissue surrounding the cervical canal to occlude the cervical canal.
